(19) United States
(12) Patent Application Publication
(10) Pub. No.: US 2019/0374790 A1
(43) Pub. Date: Dec. 12, 2019

(54) PHOTOTHERAPEUTIC DEVICE

(71) Applicant: SHARP KABUSHIKI KAISHA, Sakai City, Osaka (JP)

(72) Inventors: JUN MORI, Sakai City, Osaka (JP); HITOSHI AOKI, Sakai City, Osaka (JP)

(21) Appl. No.: 16/481,402

(22) PCT Filed: Jan. 19, 2018
(86) PCT No.: PCT/JP2018/001617

§ 371 Date: Jul. 26, 2019

(30) Foreign Application Priority Data
Jan. 26, 2017 (JP) 2017-012135

(51) Int. Cl. A61N 5/06

(52) U.S. Cl. CPC A61N 5/062; A61N 2005/007; A61N 5/0621; A61N 5/0616

(57) ABSTRACT

An uneven affected area is uniformly irradiated with light. A photoirradiation device includes a flexible substrate, a plurality of LED chips arranged in a matrix over the flexible substrate, walls having flexibility and surrounding the plurality of LED chips, and a protective resin, formed within the walls so as to cover the plurality of LED chips, that has translucency to transmit emitted light emitted by the plurality of LED chips and has flexibility. The emitted light is radiated from a region surrounded by the walls.

### TECHNICAL FIELD

The present invention relates to a phototherapeutic device that irradiates an affected area with light.

### BACKGROUND ART

Photodynamic therapy (PDT) is a method of therapy that elicits the death of diseased cells, tumors, and the like by the bactericidal activity of active oxygen and the like produced in a chemical reaction effected by irradiating, with light of a particular wavelength, a photosensitive substance having an affinity for diseased cells, tumors, and the like. Since PDT is a method of therapy that causes no damage to normal cells, it has recently attracted a lot of attention from the viewpoint of QOL (quality of life).

Further, PDT is utilized for various purposes such as the treatment of diseases such as neonatal jaundice, psoriasis, and acne, the alleviation of pain, and cosmetic purposes. For example, blue-white light is used for the treatment of neonatal jaundice, ultraviolet light for the treatment of psoriasis, and blue light, red light, and yellow light for the treatment of acne. In this way, PDT involves the use of a light source that emits light of an appropriate wavelength according to the purpose of therapy.

In recent years, PDT mainly has involved the use of a laser as a light source. Examples of reasons for that are that a laser produces monochromatic light, which can effectively excite a photosensitive substance having a narrow absorption band, that a laser is high in light intensity density, and that a laser can generate pulsed light. However, a laser beam usually forms a spot of light, which is only capable of irradiation of a narrow area and not suitable for the treatment of dermatological diseases and the like.

Further, a case has recently been reported where PDT involving the systemic administration of 5-aminolevulinic acid (ALA), which is a natural amino acid, and the use of LED light with a wavelength of 410 nm succeeded in the treatment of methicillin-resistant Staphylococcus aureus (MRSA) infected cutaneous ulcers.

ALA is a precursor substance to a porphyrin compound on a heme biosynthetic pathway and has no photosensitizing property in itself. Physiologically, production of a given amount of heme causes the biosynthesis of ALA to be inhibited by a negative feedback mechanism. However, excessive administration of exogenous ALA invalidates the negative feedback mechanism to deplete ferrochelatase, which is a rate-limiting enzyme in heme biosynthesis. As a result, large amounts of biologically-inherent porphyrin compounds, particularly protoporphyrin IX (PpIX), are accumulated in cells. PDT involving the use of ALA and LED light utilizes this PpIX as a photosensitizing substance. This method of therapy does not invite the emergence of new resistant strains of bacteria and is therefore expected to be a new method of therapy for microbial infection in modern medical care, which is having difficulties in treating resistant bacteria.

In connection with these technologies, NPL 1 discloses several PDT devices involving the use of LEDs. However, these devices are not common in Japan. A possible factor for that is that halogen lamps, xenon lamps, or metal halide lamps are common in PDT devices. These lamps are low in light emission efficiency and generate a lot of heat. Given these circumstances, PDT devices involving the use of LEDs, which are high in light emission efficiency, have been long awaited.

Furthermore, in a case where the affected area is a part having a curved surface, such as a portion of an arm a portion of a foot, a device involving the use of a lamp-type light source might force the patient into an unnatural posture, depending on whether the front side, back side, or lateral side of the part is irradiated.

Further, depending on the angle and distance of an affected area having a curved surface with respect to a device involving the use of a lamp-type light source, irradiation intensities vary from part to part that constitutes the affected area. This produces a situation where it is difficult to irradiate the affected area in its entirety with therapeutic light having a uniform irradiation intensity. Furthermore, since a device involving the use of a lamp-type light source requires a lot of auxiliary equipment such as a power source and a cooling device and is large in size, it requires a wide space for installation and is high in price.

For the expansion of use of the aforementioned PDT involving the use of LED light, there has been a demand for the realization of a photoirradiation device that can uniformly irradiate affected areas of various three-dimensional shapes and sizes with therapeutic light and, preferably, that never or hardly irradiates non-affected areas with therapeutic light.

PTL 1 discloses an alternative PDT method involving the use of ALA with high therapeutic efficacy without side effects (such as pain). PTL 1 states that PDT involving the use of ALA has a side effect called “hyperphotosensitivity” and, depending on light intensity, entails unbearable pain during therapy. A document described in PTL 1 is considered as suggesting that the side effect is developed at a certain light intensity or higher. That is, PTL 1 suggests that it is necessary that the intensity of light of a phototherapeutic device for use in PDT be confined in a certain range.

PTL 2 discloses a flexible photoirradiation device including a large number of LEDs provided as light-emitting light sources on a flexible substrate that is wound around an affected area to be irradiated with light.

### CITATION LIST

### Patent Literature

PTL 1: Japanese Unexamined Patent Application Publication No. 2014-94963 (published on May 22, 2014)
PTL 2: International Publication No. WO 2001/14012 (published on Mar. 1, 2001)

### Non Patent Literature

NPL 1: Kimura, M., “Kosen rikigaku chiryo [photodynamic therapy]”, Ushio's Technology Periodical, “Light Edge”, No. 38, <3rd special issue>, (published in October 2012)

### SUMMARY OF INVENTION

### Technical Problem

However, the aforementioned conventional technologies have the following problems.

PTL 1 does not specifically disclose what device to use. Further, the photoirradiation device disclosed in PTL 2 requires some sort of heat storage mechanism or cooling mechanism in order to cool heat produced by the light with which the affected area is irradiated. However, providing a photoirradiation device with a cooling mechanism as disclosed in PTL 2 usually complicates the mechanism to become a factor for a rise in cost and causes the photoirradiation device to lose its flexibility. This makes it difficult to uniformly irradiate an uneven affected area with light.

An aspect of the present invention was made in view of each of the foregoing problems, and it is an object of the present invention to uniformly irradiate an uneven affected area with light.

### Solution to Problem

In order to solve the foregoing problems, a phototherapeutic device according to an aspect of the present invention includes a flexible substrate, a plurality of light-emitting elements arranged in a matrix over the flexible substrate, walls having flexibility and surrounding the plurality of light-emitting elements, and a protective resin, formed within the walls so as to cover the plurality of light-emitting elements, that has translucency to transmit emitted light emitted by the plurality of light-emitting elements and has flexibility. The emitted light is radiated from a region surrounded by the walls.

### Advantageous Effects of Invention

An aspect of the present invention brings about an effect of making it possible to improve the effect of therapy by uniformly irradiating an uneven affected area with light.

### BRIEF DESCRIPTION OF DRAWINGS

FIG. 1 is a plan view showing a configuration of a front surface of a photoirradiation device according to Embodiment 1 of the present invention.

FIG. 2 is a plan view showing a configuration of a back surface of the photoirradiation device.

FIG. 3 is a cross-sectional view taken along line A-A in FIG. 1.

FIG. 4 is a cross-sectional view showing a configuration of a photoirradiation device according to a modification of Embodiment 1.

FIG. 5 is a cross-sectional view showing a configuration of a photoirradiation device according to Embodiment 2 of the present invention.

FIG. 6 is a cross-sectional view showing a configuration of a photoirradiation device according to Modification 1 of Embodiment 2.

FIG. 7 is a cross-sectional view showing a configuration of a photoirradiation device according to Modification 2 of Embodiment 2.

FIG. 8 is a plan view showing a configuration of a front surface of a photoirradiation device according to Embodiment 3 of the present invention.

FIG. 9 is a plan view showing a configuration of a front surface of a photoirradiation device according to Embodiment 4 of the present invention.

FIG. 10 is a plan view showing a configuration of a back surface of the photoirradiation device shown in FIG. 9.

FIG. 11 is a cross-sectional view taken along line B-B in FIG. 9.

FIG. 12 is a plan view showing a configuration of a front surface of a photoirradiation device according to Embodiment 5 of the present invention.

FIG. 13 is a plan view showing a configuration of a front surface of a photoirradiation device according to a modification of Embodiment 5.

### DESCRIPTION OF EMBODIMENTS

The following describes embodiments of the present invention by taking, as an example, a case where a phototherapeutic device according to an aspect of the present invention is used to perform photoirradiation therapy (hereinafter abbreviated as “phototherapy”). The following is based on the premise that a therapeutic drug has been applied to an affected area (i.e. a particular region on the skin) or has been taken in advance and is based on the premise that the affected area is kept at an appropriate distance from an LED so that the affected area can be uniformly irradiated with light in its entirety.

Further, the specific contents or the like of drugs and wavelengths of light for use in therapy are not described in detail below, as they do not affect a configuration of a phototherapeutic device according to an aspect of the present invention. Furthermore, the term “organism to be irradiated” used herein is not limited to a human but encompasses an animal.

### Embodiment 1

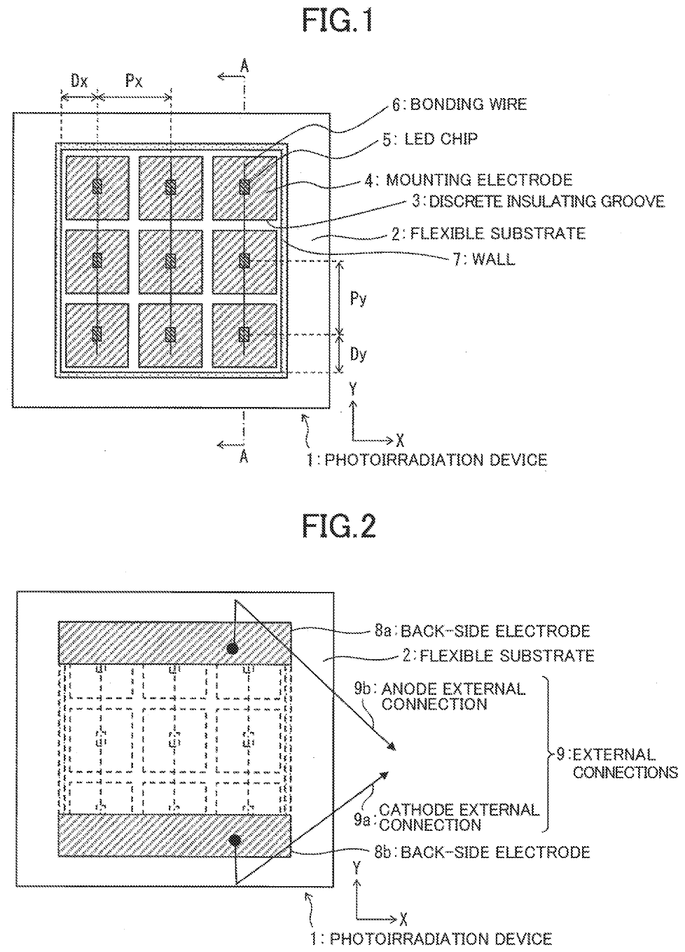

Embodiment 1 of the present invention is described below with reference to FIGS. 1 to 3.

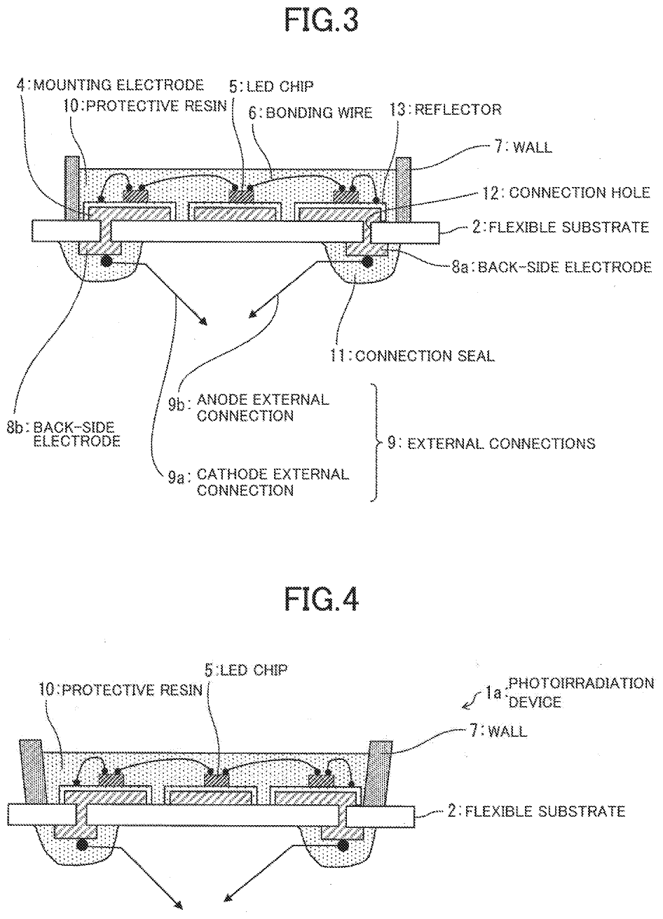

FIG. 1 is a plan view showing a configuration of a front surface of a photoirradiation device 1. FIG. 2 is a plan view showing a configuration of a back surface of the photoirradiation device 1. FIG. 3 is a cross-sectional view taken along line A-A in FIG. 1.

<Schematic Configuration of Photoirradiation Device 1>

A schematic configuration of the photoirradiation device 1 (phototherapeutic device) according to Embodiment 1 of the present invention is described with reference to FIGS. 1 to 3.

The photoirradiation device 1 is a device for, by irradiating an affected area of an organism to be irradiated (not illustrated) with LED light (light), performing phototherapy on a target illness of the organism to be irradiated.

As shown in FIGS. 1 to 3, the photoirradiation device 1 includes a flexible substrate 2, a plurality of mounting electrodes 4, a plurality of LED chips 5 (light-emitting elements), a plurality of bonding wires 6, walls 7, a pair of back-side electrodes 8a and 8b, external connections 9, a protective resin 10, connection seals 11, and reflectors 13. Usable examples of the light-emitting elements include normal laser chips and surface-emitting laser chips. In a case where such laser ships are used, it is necessary to take measures to expand a laser beam, for example, by providing the protective resin 10 with a concave lens.

The following description assumes that a surface of the photoirradiation device 1 on which the LED chips 5 (see FIG. 1) are mounted is a front surface and a surface of the photoirradiation device 1 opposite to the surface on which the LED chips 5 are mounted is a back surface.

As shown in FIG. 1, the plurality of mounting electrodes 4 are formed on a first principal surface (front surface) of the flexible substrate 2. The mounting electrodes 4 are each formed in a square shape (e.g. a regular square shape) and, when seen in a plan view, are arranged in a matrix along an X direction (first direction) and a Y direction (second direction) orthogonal to the X direction in the same plane as the X direction. Discrete insulating grooves 3 are formed between each of the mounting electrodes 4 and another, and the mounting electrodes 4 are discretely insulated from one another by the discrete insulating grooves 3. Further, the mounting electrodes 4 have their front surfaces covered with the reflectors 13.

Mounted over each of the mounting electrodes 4 is one LED chip 5 serving as a light source. Each of the LED chips 5 is connected by the bonding wires 6. The plurality of LED chips 5 are arranged in a matrix (two-dimensional array) over the flexible substrate 2.

The plurality of LED chips 5 are surrounded by the walls 7 therearound. As shown in FIG. 3, the mounting electrodes 4, the LED chips 5, and the bonding wires 6 are protected by being covered with the protective resin 10. Light emitted by the LED chips 5 is radiated outward through the protective resin 10 from a region surrounded by the walls 7.

Meanwhile, as shown in FIG. 2, the two back-side electrodes 8a and 8b are formed on a second principal surface (back surface) of the flexible substrate 2. The flexible substrate 2 has a plurality of connection holes 12 bored therethrough. Of a plurality of mounting electrodes 4 arranged in the Y direction and connected by bonding wires 6, a mounting electrode 4 at a first end is connected to the back-side electrode 8a through a connection hole 12, and a mounting electrode 4 at a second end is connected to the back-side electrode 8b through a connection hole 12.

With such a structure, LED chips 5 arranged in the Y direction are connected in series by bonding wires 6 as shown in FIG. 1. Further, series circuits of LED chips 5 forming lines in the Y direction are connected in parallel by the back-side electrodes 8a and 8b.

This allows the mounting electrodes 4 to be electrically connected to the external connections 9 via the back-side electrodes 8a and 8b. Wire connections between the external connections 9 and the back-side electrodes 8 are discretely insulated by the connection seals 11. Since the supply of a current and the application of a voltage are performed for each separate LED chip 5, each of the LED chips 5 generates heat. For this reason, the photoirradiation device 1 needs cooling or heat dissipation. In particular, cooling or heat dissipation is much needed for irradiation of a dermatological disease of a comparatively small area with light. Cooling or heat dissipation is performed, for example, by providing cooling means on the back surface of the flexible substrate 2 or pasting a thermally-conductive flexible material or a heat-dissipative flexible material to the back surface of the flexible substrate 2.

### <Configuration of Components of Photoirradiation Device 1>

Next, the constituent elements of the photoirradiation device 1 are described in detail.

(Flexible Substrate 2)

The flexible substrate 2 is an insulating substrate having flexibility and, for example, is formed by an insulating film of polyimide or the like. Note, however, that a material of the flexible substrate 2 does not need to be limited to polyimide, and any insulating material that has the required strength and flexibility may be used. As the flexible substrate 2, a film of fluororesin, silicone resin, polyethylene terephthalate, or the like as well as a polyimide film may be used. Further, the flexible substrate 2 may be made of any of various materials such as a highly reflective resin film obtained by applying resin containing a white pigment (such as white resin or a white resist) to a front surface of any of these films, a highly reflective resin film mixed with a white pigment, or a liquid crystal polymer film.

Phototherapy is provided to affected areas of various shapes, sizes, and areas. For this reason, there is no particular limitation on the size or shape of the flexible substrate 2. The flexible substrate 2 only needs to have such a size as to cover an affected area. When the photoirradiation device 1 has such a size as to cover only an affected area and irradiate it with light, the patient is less restricted and the burden on the patient can be reduced to the minimum.

The photoirradiation device 1 is suitably used for a local disease of a comparatively small area of approximately several centimeters. It is desirable that the flexible substrate 2 be formed in a size corresponding to this local disease.

There is no particular limitation on the thickness of the flexible substrate 2, provided it has the required strength and flexibility. While the present embodiment uses a film having a thickness of 50 μm, a film having a different thickness may be used with no problem.

(Mounting Electrodes 4 and Reflectors 13)

In order to reduce a loss during photoirradiation, it is necessary to minimize an energy loss caused by a reflection of an electrode material on the flexible substrate 2. Therefore, it is desirable that the electrode material be low in resistance and high in surface reflectance. Specifically, it is desirable that the electrode material have a total luminous flux reflectance of at least 80%, desirably 90% or higher.

The term “total luminous flux reflectance” here refers not to the reflectance of a specular reflection but to the proportion of light energy obtained by integrating all of reflected light diffusedly reflected to energy of incident light.

For this reason, the reflectors 13, which are formed on at least the front surfaces of the mounting electrodes 4 on the front side of the flexible substrate 2, are made of a reflective material (hereinafter referred to as “high-reflectance material”) having a total luminous flux reflectance of 80% or higher, desirably a high-reflectance material having a total luminous flux reflectance of 90% or higher. This makes it possible to reflect as much as possible of light reflected from the affected area, return the light to the affected area, and thereby reduce a loss of the light to the minimum.

The high-reflectance material may be a regular reflective material or may be a diffuse reflective material. In the present embodiment, the reflectors 13 are each formed by a copper wire whose front surface is plated with silver. The reflectors 13 are not limited to this but may be formed by a material such as aluminum.

(LED Chips 5 and Bonding Wires 6)

The LED chips 5 are selected according to the purpose of therapy. The photoirradiation device 1 is here applied to “methicillin-resistant Staphylococcus aureus (MRSA) infected cutaneous ulcer therapy” by using LED chips 5 (with a peak wavelength of 410 nm) that emit gallium nitride blue-violet light. In other uses, optimum LED chips 5, such as ultraviolet LEDs, blue LEDs, or green LEDs based on gallium nitride (AlInGaN) LEDs; red LEDs, yellow LEDs, or green LEDs based on quaternary (AlGaInP) LEDs; or GaAs infrared LEDs, can be selected for different purposes. Alternatively, it is also possible to combine a plurality of LED chips 5 of different wavelength bands.

An affected area of a certain size can be better uniformly irradiated with light as in the case of phototherapy by arranging a large number of comparatively small LED chips 5 than by using a small number of high-power LED chips 5. In the present embodiment, nine LED chips 5 each measuring 440 μm×550 μm in size are mounted over the flexible substrate 2. These LED chips 5 emit blue-violet light.

As shown in FIG. 1, the LED chips 5 are arranged in a 3-by-3 matrix along the X and Y directions. As shown in FIG. 1, assuming that Px denotes the pitch between LED chips 5 adjacent to each other in the X direction Py denotes the pitch between LED chips 5 adjacent to each other in the Y direction, the LED chips 5 are arranged at substantially regular intervals (Px, Py) in a matrix.

In the present embodiment, the LED chips 5 are arrayed parallel to each side of the square (regular square) flexible substrate 2. Further, the pitch between LED chips 5 adjacent to each other in the X or Y direction represents the distance between the centers of LED chips 5 adjacent to each other in the X or Y direction.

By thus arranging the LED chips 5 at substantially regular intervals (Px, Py) in a two-dimensional array in the photoirradiation device 1, the uniformity of photoirradiation intensity within the photoirradiation device 1 can be improved.

In general, Px=Py; however, there is a case where light output distributions may vary between the X and Y directions depending on the shapes of the LED chips 5. In this case, it is desirable that the pitches (Px, Py) between LED chips 5 vary between the X and Y directions. For example, an LED chip 5 of an elongate shape tends to easily emit light in a direction perpendicular to a long side thereof and emit little light in a direction perpendicular to a short side thereof. Further, in the case of LED chips 5 whose long sides are parallel, for example, to the X direction, it is desirable that Px<Py. For the highest level of simplification, it is desirable that almost regular square LED chips 5 be used and Px=Py. Note here that the aforementioned tendency may be affected by an arrangement of electrodes of the LED chips 5. For this reason, it is desirable that optimizations be performed based on the actual light emission properties of the LED chips 5.

In the present embodiment, the average pitch between LED chips 5 ranges from approximately 5 mm to 10 mm. An LED chip 5 of this size is highest in light emission efficiency when it is an LED chip of the most common structure in which a nitride semiconductor layer is epitaxially grown on a sapphire substrate and a cathode electrode and an anode electrode (both not illustrated) are formed on the same plane.

In the present embodiment, the aforementioned LED chips 5 each in of which a cathode electrode and an anode electrode are formed on the same plane are bonded onto the mounting substrate 5 by a transparent die bonding paste. Of a plurality of LED chips 5 arranged in the Y direction, an LED chip 5 connected to the back-side electrode 8a has its anode electrode connected to the mounting electrode 4 by a bonding wire 6. Further, of a plurality of LED chips 5 arranged in the Y direction, an LED chip 5 connected to the back-side electrode 8b has its cathode electrode connected to the mounting electrode 4 by a bonding wire 6. Further, as shown in FIGS. 1 and 3, the cathode electrode of one of two LED chips 5 adjacent to each other in the Y direction and the anode electrode of the other of the two LED chips 5 are connected to each other by a bonding wire 6.

The bonding wires 6 are formed by gold (gold bonding wires). Note, however, that the bonding wires 6 do not necessarily need to be made of gold but may be formed by publicly-known bonding wires made of silver, aluminum, or the like.

In a case where quaternary (AlGaInP) LEDs or GaAs infrared LEDs are used as the LED chips 5, the LED chips 5 have a so-called upper and lower electrode structure. For this reason, such an upper and lower electrode structure of the LED chips 5 makes the structure of connection to the mounting electrodes 4 different from the structure of connection shown in FIG. 3. Specifically, a lower surface of an LED chip 5 that serves as a lower electrode of the LED chip 5 is bonded to a mounting electrode 4 by an electrical conducting material such as a silver paste, and an upper electrode is bonded by a bonding wire 6 to a mounting electrode 4 which is different from that on which the LED chip 5 is mounted.

(Back-Side Electrodes 8a and 8b and External Connections)

The external connections 9 are wiring connections for connecting to an external power source. The photoirradiation device 1 has its back-side electrodes 8a and 8b supplied with electric power via the external connections 9. This causes the LED chips 5 to be supplied with a current via the mounting electrodes 4 from the back-side electrodes 8a and 8b.

In the present embodiment, as shown in FIGS. 2 and 3, the external connections 9 are provided on a back surface side of the flexible substrate 2. The external connections 9 are connected to the back-side electrodes 8a and 8b by soldering or the like. The back-side electrodes 8a and 8b are connected to some of the front-side mounting electrodes 4 via the connection holes 12. This causes back-side electrodes 8a and 8b and the mounting electrodes 4 to be electrically connected to each other. Therefore, the external connections 9 are electrically connected to the mounting electrodes 4 via the back-side electrodes 8a and 8b.

The external connections 9 each include, for example, a lead wire and a connector or the like for connecting the lead wire to the flexible substrate 2. Further, for enhanced convenience of connection to the power source, it is preferable that the external connections 9 be each configured to be terminated by a socket, a plug, or the like so as to be able to be easily connected to the power source.

Accordingly, the configuration shown in FIGS. 2 and 3 includes lead wires as the external connections 9. However, the configuration in which the external connections 9 are each a lead wire is merely an example, and in actuality, it is needless to say that the flexible substrate 2 may be provided with a connector or the like for connecting a lead wire.

Further, as shown in FIGS. 2 and 3, the external connections 9 include a cathode external connection 9a and an anode external connection 9b. The cathode external connection 9a is connected to the back-side electrode 8b, and the anode external connection 9b is connected to the back-side electrode 8a.

It is preferable that the back-side electrodes 8a and 8b be covered with the connection seals 11, made of insulating resin, which cover the wire connections between the external connections 9 and the back-side electrodes 8a and 8b, respectively. By thus covering the back-side electrodes 8a and 8b (wire connections) with the connection seals 11, respectively, the back-side electrodes 8a and 8b can be discretely insulated from each other and the insulation properties of a back surface of the photoirradiation device 1 can be secured.

(Walls 7)

The walls 7 are formed on a front surface side of the flexible substrate 2. Further, the walls 7 are formed to be higher than the LED chips 5 and the bonding wires 6, which are located within the walls 7. A region surrounded by the walls 7 (within the walls 7) is filled with the protective resin 10.

The walls 7 are formed by the following method. White resin, e.g. Shin-Etsu Silicone's KER-2000-DAM, is charged into a syringe and applied onto the flexible substrate 2 using a coating robot, e.g. Musashi Engineering's SHOTMASTER 300SX. In the present embodiment, the walls 7 have a height of 0.6 mm. Then, the white resin is cured by heating at 110° C. for one hour.

After being cured, the walls 7 have the flexibility, for example, to have a rubber hardness of approximately 10 to 30. This makes it possible to reduce the risk of impairing the flexibility of the flexible substrate 2. This allows the photoirradiation device 1 to have flexibility. This enables the photoirradiation device 1 to conform to an uneven affected area. Further, it is important that the walls 7 and the flexible substrate 2 have high adhesiveness to each other. Since the walls 7 and the protective resin 10 are integrated with each other, peeling of the protective resin 10 can be prevented.

The walls 7 have light reflectivity by being formed by a reflective material. This makes it possible to cause light from the LED chips 5 to be reflected by the walls 7 and extract the light through the protective resin 10. This also makes it possible to substantially prevent a region other than the region surrounded by the walls 7 from being irradiated with light.

As shown in FIG. 1, the walls 7 are formed at a substantially constant distance Dx from the closest LED chips 5 in the X direction and at a substantially constant distance Dy from the closes LED chips 5 in the Y direction. It is desirable that the distances Dx and Dy satisfy the following conditions.

0.5Dx≤Px≤4×Dx

0.5Dy≤Py≤4×Dy

Specifically, the foregoing conditions are to satisfy both the following first and second conditions. The first condition is that the average distance (distance Px) between LED chips 5 adjacent to each other in the X direction falls within a range of 0.5 to 4 times the average distance (distance Dx) between an LED chip 5 closest to the walls 7 in the X direction and the walls 7. The second condition is that the average distance (distance Py) between LED chips 5 adjacent to each other in the Y direction falls within a range of 0.5 to 4 times the average distance (distance Dy) between an LED chip 5 closest to the walls 7 in the Y direction and the walls 7.

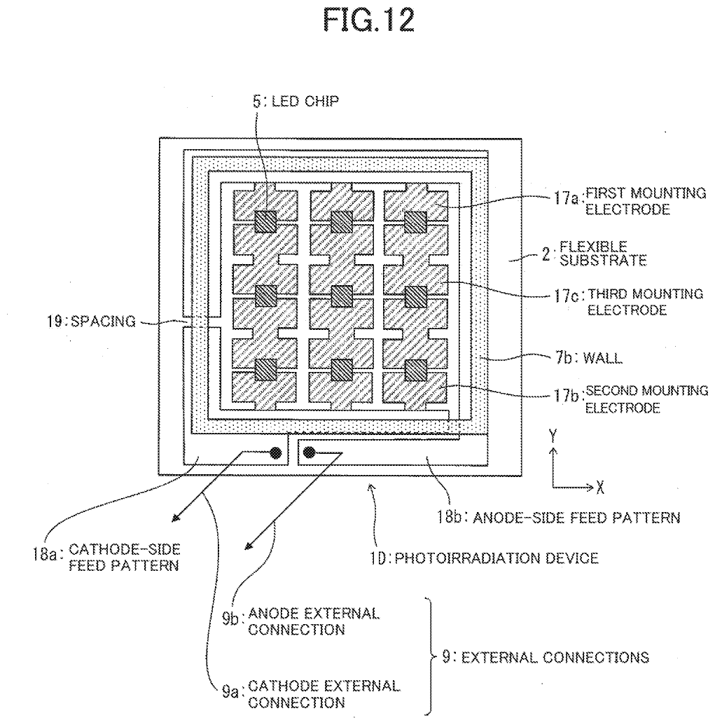

By satisfying the foregoing first and second conditions, the in-plane uniformity of intensity of light can be improved. As for walls 7a (see FIG. 8) of Embodiment 3 to be described below and walls 7b (see FIG. 12) of Embodiment 5 to be described below, the foregoing first and second conditions are satisfied.

The following method is not the only method for forming the walls 7 but may be replaced by a method such as bonding of rubber sheets. Further, it is also possible to enhance the adhesiveness between the walls 7 and the flexible substrate 2 by varying the surface roughness of a part of the flexible substrate 2 that adheres to the walls 7.

(Protective Resin 10)

The protective resin 10 has its front surface located at a lower level than upper ends of the walls 7. Accordingly, the height of the walls 7 is determined according to the maximum height of the protective resin 10. As the protective resin 10 becomes thicker, the photoirradiation device 1 can emit light with higher in-plane uniformity. However, the transmittance of the protective resin 10 is not 100%. For this reason, for reduction of individual variations in the intensity and in-plane uniformity of light that the photoirradiation device 1 emits, it is important to make the thickness of the protective resin 10 as constant as possible. By controlling at least either the coating volume or coating time of the protective resin 10 and leaving the protective resin 10 on a horizontal desk for approximately ten minutes, in-plane uniformity in thickness of the protective resin 10 is achieved. This makes it possible to reduce individual variations in the thickness of the protective resin 10 and makes it possible to reduce individual variations in the intensity and in-plane uniformity of light that the photoirradiation device 1 emits.

Usable examples of the protective resin 10 include silicone resin and epoxy resin. It is desirable that the protective resin 10 be transparent so as to have the translucency to transmit light (emitted light) emitted by the LED chips 5. For this reason, it is desirable that the protective resin 10 have a transmittance of 80% or higher. This allows the photoirradiation device 1 to consume less electric power and by extension makes it possible to reduce the amount of heat that is generated by the photoirradiation device 1.

Further, the protective resin 10 has flexibility. This allows the photoirradiation device 1 to have flexibility and be able to conform to an uneven body part.

The protective resin 10 may contain a known wavelength conversion material such as a phosphor.

<Effects of Photoirradiation Device 1>

The photoirradiation device 1 includes the flexible substrate 2 as a substrate on which to mount the LED chips 5, and the LED chips 5 are covered with the protective resin 10 having translucency and flexibility. This makes it possible to reduce the heat of light with which an affected area is irradiated, as the protective resin 10 absorbs heat generated by emitted light from the LED chips 5. Accordingly, the photoirradiation device 1 does not need to include a complex cooling structure such as that disclosed in PTL 2 and does not impair the flexibility of the flexible substrate 2. This makes it possible to uniformly irradiate an uneven affected area with light in conformance with the affected area.

Further, mounting electrodes 4 adjacent to each other are placed at an interval from each other. Such a structure makes it possible not only to firmly mount the LED chips 5 over the flexible substrate 2 but also to secure the flexibility of the flexible substrate 2 in the X and Y directions.

[Modification]

A modification of Embodiment 1 is described with reference to FIG. 4. FIG. 4 is a cross-sectional view showing a configuration of a photoirradiation device 1a according to the modification.

In the photoirradiation device 1 described above, the walls 7 are formed so as to stand up perpendicularly to the front surface of the flexible substrate 2. Light emitted by the LED chips 5 is reflected by inner wall surfaces of the walls 7 and radiated outward through the protective resin 10. However, since the inner wall surfaces of the walls 7 are perpendicular to the flexible substrate 2, the light thus reflected travels sideward at a great inclination with respect to light traveling in straight lines from the LED chips 5. For this reason, a portion of the emitted light from the LED chips 5 cannot be utilized.

On the other hand, as shown in FIG. 4, in the photoirradiation device 1a according to the present modification, the walls 7 are inclined so that the area surrounded by the inner walls of the walls 7 becomes wider toward the upper ends. This causes light emitted by the LED chips 5 to, once reflected by the walls 7, travel at a small inclination with respect to light traveling in straight lines from the LED chips 5. Therefore, the reflected light can be applied to an affected area, too. Accordingly, the reflected light from the walls 7 can be utilized for therapy, too.

### Embodiment 2

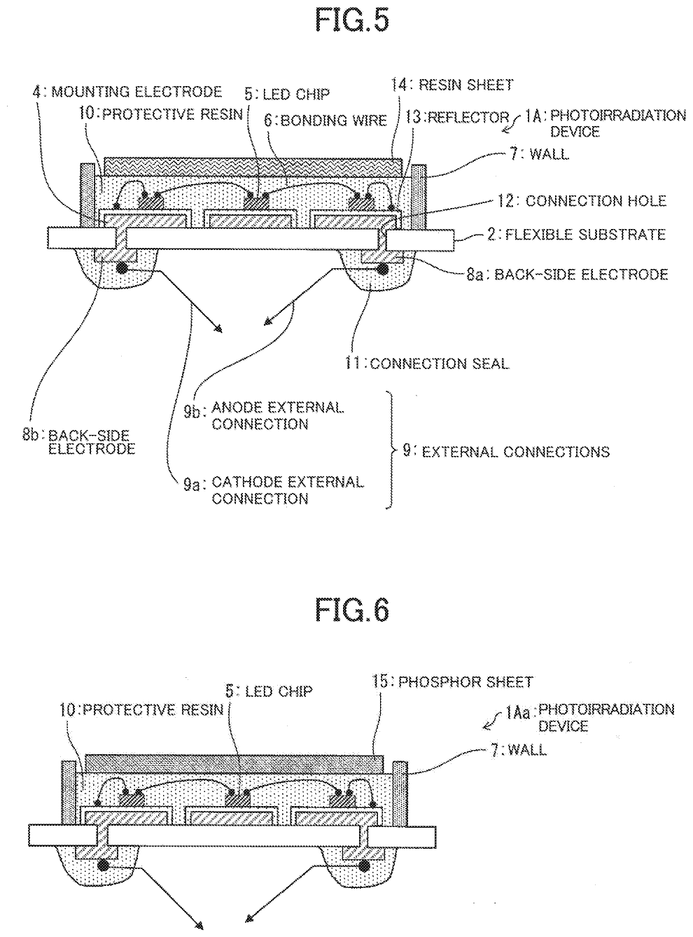

Embodiment 2 of the present invention is described below with reference to FIGS. 5 to 7. For convenience of explanation, members having the same functions as those described in Embodiment 1 are given the same signs and are not described below. Further, the present embodiment describes points of difference from Embodiment 1.

FIG. 5 is a cross-sectional view showing a configuration of a photoirradiation device 1A according to the present embodiment.

As shown in FIG. 5, the photoirradiation device 1A includes a flexible substrate 2, a plurality of mounting electrodes 4, a plurality of LED chips 5, a plurality of bonding wires 6, walls 7, a pair of back-side electrodes 8a and 8b, external connections 9, a protective resin 10, connection seals 11, and reflectors 13, as with the photoirradiation device 1 of Embodiment 1. Further, the photoirradiation device 1A further includes a resin sheet 14.

The resin sheet 14 is a sheet member made of resin and formed so as to overlap the front surface of the protective rein 10. The resin sheet 14 is composed of resin having translucency and biocompatibility. Further, although not illustrated, in a case where the front surface of the protective resin 10 is at a lower level than the upper ends of the walls 7, the resin sheet 14 is formed so as to have such a thickness that a front surface of the resin sheet 14 is located at a higher level than the upper ends of the walls 7.

This brings the resin sheet 14 into direct contact with not the upper ends of the walls 7 but an affected area. Further, since the resin sheet 14 has biocompatibility, it can favorably maintain contactability with the affected area.

It is desirable that the resin sheet 14 have flexibility. This allows the photoirradiation device 1 to have flexibility and be able to irradiate an uneven affected area with light in conformance with the affected area.

[Modification 1]

Modification 1 of Embodiment 2 is described with reference to FIG. 6. FIG. 6 is a cross-sectional view showing a configuration of a photoirradiation device 1Aa according to Modification 1.

As shown in FIG. 6, the photoirradiation device 1Aa according to Modification 1 includes, instead of the aforementioned resin sheet 14, a phosphor sheet 15 (wavelength conversion sheet) made of resin.

The phosphor sheet 15 is composed of resin having translucency and biocompatibility and has a large number of fine phosphors (wavelength conversion materials) dispersed therein. Further, as with the resin sheet 14, in a case where the front surface of the protective resin 10 is at a lower level than the upper ends of the walls 7, the phosphor sheet 15 is formed so as to have such a thickness that a front surface of the phosphor sheet 15 is located at a higher level than the upper ends of the walls 7.

Light emitted by the LED chips 5 and having passed through the protective resin 10 illuminates the phosphors while directly passing through the inside of the phosphor sheet 15. By being excited by the illuminating light, the phosphors emit light that is different in wavelength from the emitted light. Light produced by a mixture of the light having passed through the inside of the phosphor sheet 15 and the light whose wavelength has been converted by the phosphors is radiated from the phosphor sheet 15. This makes it possible to irradiate an affected area with light having a wavelength needed for phototherapy.

It is desirable that the phosphor sheet 15 have flexibility, as with the resin sheet 14. This allows the photoirradiation device 1Aa to have flexibility and be able to irradiate an uneven affected area with light in conformance with the affected area.

Alternatively, a plurality of the phosphor sheets 15 may be provided so as to overlap each other. This makes it possible to vary the emission spectrum of the photoirradiation device 1Aa. This makes it possible to select and customize an emission spectrum of the photoirradiation device 1Aa that is most suitable for therapy.

Further, any number of resin sheets 14 may be provided over the phosphor sheet 15 so as to overlap each other. This makes it possible to enhance the in-plane uniformity of light that the LED chips 5 emit.

It is desirable that the resin sheets 14 have heat insulating properties as well as insulation properties. This makes it possible to block an affected area from heat emitted by the photoirradiation device 1A and makes it possible to favorably maintain comfortability during therapy.

[Modification 2]

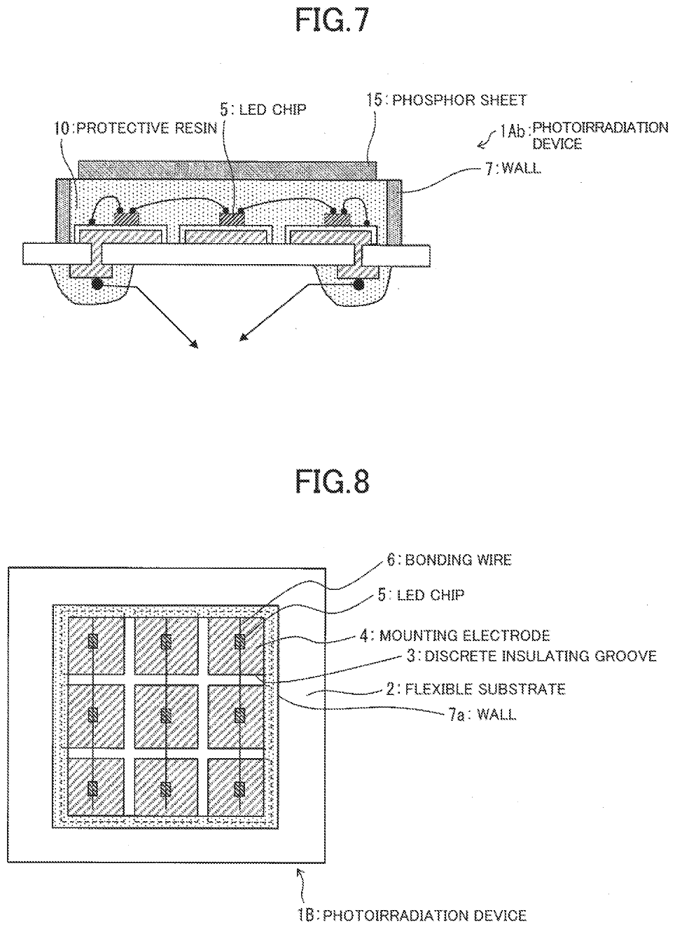

Modification 2 of Embodiment 2 is described with reference to FIG. 7. FIG. 7 is a cross-sectional view showing a configuration of a photoirradiation device 1Ab according to Modification 2.

As shown in FIG. 7, the photoirradiation device 1Ab according to Modification 2 differs from the aforementioned photoirradiation device 1Aa in that the front surface of the protective resin 10 is located at substantially the same level as the upper ends of the walls 7. Further, although not illustrated, the front surface of the protective resin 10 may be located at a higher level than the upper ends of the walls 7.

In the foregoing configuration, the walls 7 can be used for positioning of the phosphor sheet 15 even if the front surface of the protective resin 10 is located at the same level as or a higher level than the upper ends of the walls 7.

In the photoirradiation device 1Ab, the phosphor sheet 15 may be replaced by a resin sheet 14, and any number of resin sheets 14 may be joined on top of each other. This makes it possible to enhance the in-plane uniformity of light that the LED chips 5 emit.

It should be noted that Modifications 1 and 2 described above are also applicable to Embodiments 3 to 5 to be described below.

### Embodiment 3

Embodiment 3 of the present invention is described below with reference to FIG. 8. For convenience of explanation, members having the same functions as those described in Embodiments 1 and 2 are given the same signs and are not described below. Further, the present embodiment describes points of difference from Embodiments 1 and 2. FIG. 8 is a plan view showing a configuration of a front surface of a photoirradiation device 1B according to the present embodiment.

As shown in FIG. 8, the photoirradiation device 1B includes a flexible substrate 2, a plurality of mounting electrodes 4, a plurality of LED chips 5, a plurality of bonding wires 6, a pair of back-side electrodes 8a and 8b, external connections 9, a protective resin 10, connection seals 11, and reflectors 13, as with the photoirradiation device 1 of Embodiment 1. Further, the photoirradiation device 1B includes walls 7a.

The walls 7a are formed by the same material and forming method as the walls 7 of the photoirradiation device 1. Note, however, that unlike the walls 7, the walls 7a are formed not only on the front surface of the flexible substrate 2 but also to partially overlap the mounting electrodes 4. Specifically, the walls 7a are formed so as to surround all of the LED chips 5, as with the walls 7; however, parts of inner edges all around the walls 7a are formed over the front surfaces of parts of mounting electrodes 4 located close to the walls 7a. Further, the mounting electrodes 4 have their front surfaces constituted by a metal material.

Such a structure of the walls 7a causes the walls 7a to be fixed to the mounting electrodes 4 and the flexible substrate 2 with high adhesive strength. The adhesive strength is higher than the adhesive strength with which the walls 7 are fixed to the flexible substrate 2 in the photoirradiation device 1 of Embodiment 1 and the photoirradiation devices 1 and 1A of Embodiment 2, which have the walls 7 formed only on the flexible substrate 2.

The surface roughness of the mounting electrodes 4 is a vital component in the determination of the adhesive strength between the mounting electrodes 4 and the walls 7a. It is also possible to enhance the adhesive strength between the mounting electrodes 4 and the walls 7a by appropriately adjusting the surface roughness of the mounting electrodes 4. Since the mounting electrodes 4 have their front surfaces constituted by a metal material, the surface roughness may be adjusted by sandpapering or the like.

Further, the walls 7a are smaller in size than the walls 7. This makes it possible to make the photoirradiation device 1B smaller in size than the aforementioned photoirradiation devices 1 and 1A. This brings a cost advantage.

### Embodiment 4

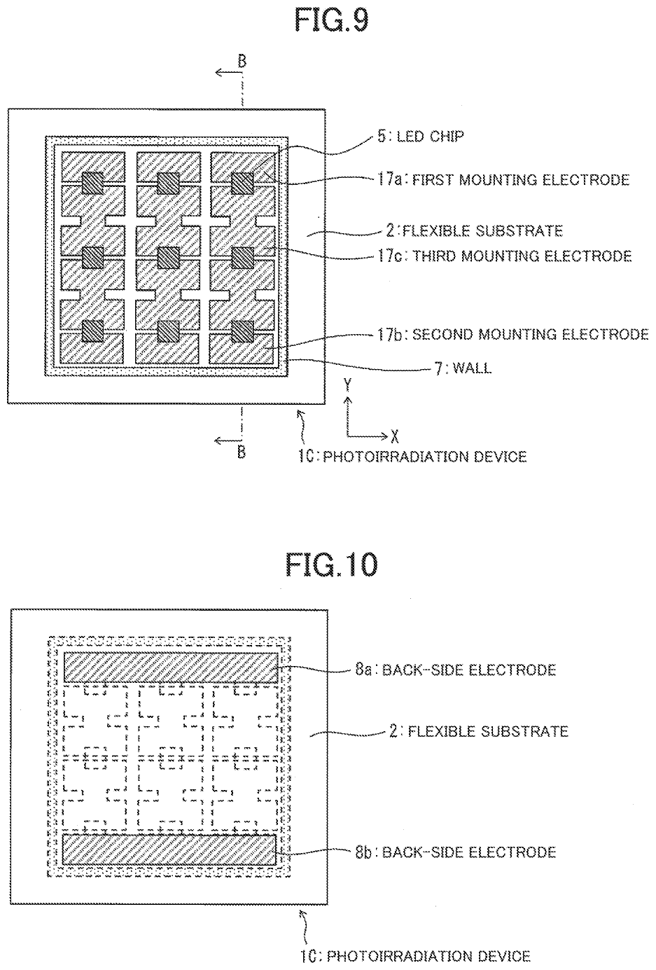

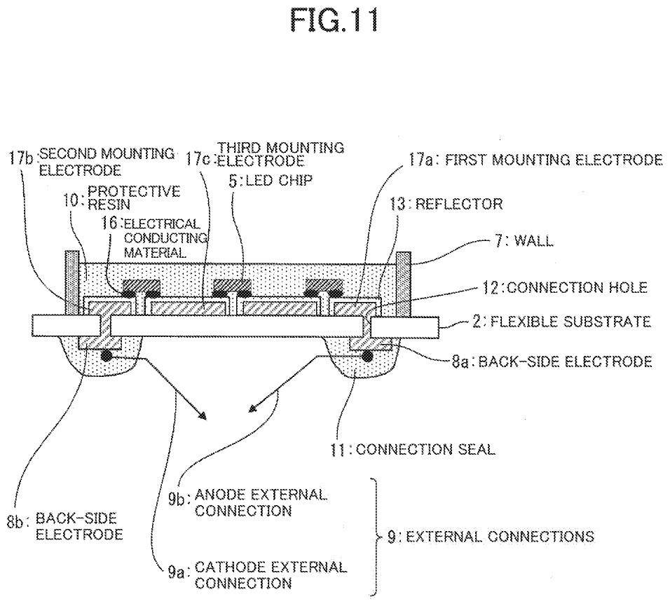

Embodiment 4 of the present invention is described below with reference to FIGS. 9 to 11. For convenience of explanation, members having the same functions as those described in Embodiments 1 to 3 are given the same signs and are not described below. Further, the present embodiment describes points of difference from Embodiment 1. FIG. 9 is a plan view showing a configuration of a front surface of a photoirradiation device 1C according to the present embodiment. FIG. 10 is a plan view showing a configuration of a back surface of the photoirradiation device 1C. FIG. 11 is a cross-sectional view taken along line B-B in FIG. 9.

As shown in FIGS. 9 to 11, the photoirradiation device 1C includes a flexible substrate 2, a plurality of LED chips 5, walls 7, a pair of back-side electrodes 8a and 8b, external connections 9, a protective resin 10, connection seals 11, and reflectors 13, as with the photoirradiation device 1 of Embodiment 1. Further, the photoirradiation device 1C includes an electrical conducting material 16, a first mounting electrode 17a, a second mounting electrode 17b, and a third mounting electrode 17c.

Each of the LED chips 5 according to the present embodiment has a size of approximately 1 cm per side and has an anode electrode and a cathode electrode on a lower surface thereof. The LED chips 5 are mounted over the first mounting electrode 17a, the second mounting electrode 17b, and the third mounting electrode 17c by flip-chip mounting. The first mounting electrode 17a, the second mounting electrode 17b, and the third mounting electrode 17c are formed on the front surface of the flexible substrate 2. The first mounting electrode 17a and the second mounting electrode 17b, forming rectangular shapes, are placed so that their long sides extend along the X direction. Meanwhile, the third mounting electrode 17c, formed in a rectangular shape constricted in the middle, is placed so that the constricted sides extend along the Y direction.

The first mounting electrode 17a is placed opposite the back-side electrode 8a. The second mounting electrode 17b is placed opposite the back-side electrode 8b. The same numbers of the first and second mounting electrodes 17a and 17b are provided opposite the back-side electrodes 8a and 8b, respectively, as the number of LED chips 5 arranged in the X direction. Further, the first mounting electrode 17a, the second mounting electrode 17b, and the third mounting electrode 17c are arranged so that those of them which are adjacent to each other in the X direction are placed at predetermined intervals from each other.

The number of third mounting electrodes 17c provided in each column along the Y direction between first and second mounting electrodes 17a and 17b facing each other in the Y direction is smaller by one than the number of LED chips 5 arranged in the Y direction. First and third mounting electrodes 17a and 17c adjacent to each other in the Y direction, second and third mounting electrodes 17b and 17c adjacent to each other in the Y direction, and third mounting electrodes 17c adjacent to each other in the Y direction are placed at predetermined intervals from each other.

As shown in FIGS. 9 and 11, over the first and third mounting electrodes 17a and 17c adjacent to each other in the Y direction, over the second and third mounting electrodes 17b and 17c adjacent to each other in the Y direction, and over the third mounting electrodes 17c adjacent to each other in the Y direction, LED chips 5 are mounted via the electrical conducting material 16, respectively.

The LED chip 5 mounted over the first and third mounting electrodes 17a and 17c adjacent to each other in the Y direction has its anode electrode connected to the first mounting electrode 17a and its cathode electrode connected to the third mounting electrode 17c. The LED chip 5 mounted over the second and third mounting electrodes 17b and 17c adjacent to each other in the Y direction has its cathode electrode connected to the second mounting electrode 17b and its anode electrode connected to the third mounting electrode 17c. The LED chip 5 mounted over the third mounting electrodes 17c adjacent to each other in the Y direction has its anode electrode connected to the third mounting electrode 17c placed beside the first mounting electrode 17a and its cathode electrode connected to the third mounting electrode 17c placed beside the second mounting electrode 17b.

Such a mounting structure of the LED chips 5 causes LED chips 5 arranged in the Y direction to be connected in series via a first mounting electrode 17a, a second mounting electrode 17b, and third mounting electrodes 17c as shown in FIG. 9. Further, as shown in FIG. 10, series circuits of LED chips 5 forming lines in the Y direction are connected in parallel by the back-side electrodes 8a and 8b.

In the photoirradiation device 1C thus configured, the first mounting electrodes 17a, the second mounting electrodes 17b, and the third mounting electrodes 17c also function as wires by which the LED chips 5 are connected in series. This makes it unnecessary to include the bonding wires 6 that the photoirradiation device 1 of Embodiment 1 includes, thus removing restrictions placed on a range of movement by the bonding wires 6. This makes it possible to enhance the flexibility of the photoirradiation device 1C.

First mounting electrodes 17a adjacent to each other in the X direction, second mounting electrodes 17b adjacent to each other in the X direction, and third mounting electrodes 17c adjacent to each other in the X direction are placed at intervals from each other. First and third mounting electrodes 17a and 17c adjacent to each other in the Y direction are placed at an interval from each other. Further, second and third mounting electrodes 17b and 17c adjacent to each other in the Y direction are placed an interval from each other. Further, third mounting electrodes 17c adjacent to each other in the Y direction are placed at an interval from each other. Moreover, the third mounting electrodes 17c are constricted in the middle, and these constricted parts give flexibility. Such a structure makes it possible not only to firmly mount the LED chips 5 over the flexible substrate 2 but also to secure the flexibility of the flexible substrate 2 in the X and Y directions.

As shown in FIG. 11, the LED chips 5 are electrically connected to the first mounting electrodes 17a, the second mounting electrodes 17b, and the third mounting electrodes 17c via the electrical conducting material 16. It is desirable that after being cured, the electrical conducting material 16 have high adhesive strength with the second mounting electrodes 17b and the third mounting electrodes 17c and have flexibility. This allows the photoirradiation device 1 to have flexibility and be able to easily conform to an uneven affected area.

Although the photoirradiation device 1C includes the walls 7, as with the photoirradiation device 1 of Embodiment 1, the photoirradiation device 1C may alternatively include walls 7a instead of the walls 7, as with the photoirradiation device 1B of Embodiment 3.

It should be noted that the configuration of the modification of Embodiment 1, Modification 1 of Embodiment 2, or Modification 2 of Embodiment 2 is also applicable to the photoirradiation device 1C of the present embodiment.

### Embodiment 5

Embodiment 5 of the present invention is described below with reference to FIG. 12. For convenience of explanation, members having the same functions as those described in Embodiments 1 to 4 are given the same signs and are not described below. Further, the present embodiment describes points of difference from Embodiments 1 and 4. FIG. 12 is a plan view showing a configuration of a front surface of a photoirradiation device 1D according to the present embodiment.

As shown in FIG. 12, the photoirradiation device 1D includes a flexible substrate 2, a plurality of LED chips 5, external connections 9, a protective resin 10, connector seals 11 (not illustrated), and reflectors 13 (not illustrated), as with the photoirradiation device 1 of Embodiment 1. Further, the photoirradiation device 1D includes an electrical conducting material 16, a first mounting electrode 17a, a second mounting electrode 17b, and a third mounting electrode 17c, as with the photoirradiation device 1C. Furthermore, the photoirradiation device 1D includes a cathode-side feed pattern 18a (feeder wire) and an anode-side feed pattern 18b (feeder wire). Further, the photoirradiation device 1D includes walls 7b.

As with the LED chips 5 according to Embodiment 4, each of the LED chips 5 according to the present embodiment has a size of approximately 1 cm per side and has an anode electrode and a cathode electrode on a lower surface thereof.

The cathode-side feed pattern 18a and the anode-side feed pattern 18b are formed on the front surface of the flexible substrate 2. The anode-side feed pattern 18b is formed in an area surrounding three corners of a square region around the first mounting electrodes 17a, the second mounting electrodes 17b, and the third mounting electrodes 17c. Meanwhile, the cathode-side feed pattern 18a is formed in an area surrounding the remaining one corner of the square region. The anode-side feed pattern 18b is connected to all of the first mounting electrodes 17a in a portion extending in the X direction. The cathode-side feed pattern 18a has a portion extending parallel to the anode-side feed pattern 18b in the X direction, and is connected to all of the second mounting electrodes 17b in this portion.

The cathode-side feed pattern 18a and the anode-side feed pattern 18b have their respective first ends facing each other in the X direction at an interval on a side close to the second mounting electrodes 17b. The cathode-side feed pattern 18a has its first end connected to the cathode external connection 9a of the external connections 9. The anode-side feed pattern 18b has its first end connected to the anode external connection 9b of the external connections 9.

The walls 7b are formed by the same material and forming method as the walls 7 of the photoirradiation device 1. Note, however, that unlike the walls 7, the walls 7b are formed on the cathode-side feed pattern 18a and the anode-side feed pattern 18b so as to surround all of the first, second, and third mounting electrodes 17a, 17b, and 17c and all of the LED chips 5.

Alternatively, the walls 7b may be formed on the front surface of the flexible substrate 2 so as to surround the cathode-side feed pattern 18a and the anode-side feed pattern 18b.

In the photoirradiation device 1D thus configured, the external connections 9 are provided in positions spatially closer to each other than in the photoirradiation device 1 of Embodiment 1. With this, in a state where the external connections 9 are connected to the power source, a relative displacement in position of the photoirradiation device 1D and the power source, if any, does not make a great difference in force application between the cathode external connection 9a and the anode external connection 9b. This makes it possible to prevent the photoirradiation device 1D from being subjected to unnecessary force by being pulled toward the power source via either the cathode external connection 9a or the anode external connection 9b. This enables the photoirradiation device 1D to more easily conform to an uneven body part by avoiding being deformed by the unnecessary force.

Further, as shown in FIG. 12, the cathode-side feed pattern 18a and the anode-side feed pattern 18b have their respective second ends facing each other in the Y direction, with a spacing 19 formed between the second ends. This makes it possible to mount a zener diode or the like in the spacing 19. This makes it possible to protect the LED chips 5 when backward voltage or static electricity is generated unexpectedly.

Furthermore, unlike the photoirradiation device 1, the photoirradiation device 1D does not need to include electrodes such as the back-side electrodes 8a and 8b on the back surface of the flexible substrate 2. This makes it unnecessary to provide the flexible substrate 2 with connection holes 12. This brings a cost advantage.

The photoirradiation device 1D may have a metal plate formed on the back surface of the flexible substrate 2. This makes it possible to increase the mechanical strength of the photoirradiation device 1D. By having a pattern appropriately formed thereon, this metal plate makes it possible to reduce stress put on the electrical conducting material 16 (see FIG. 11) in a state of conforming to an affected area having no flat surface. Heat generated by each LED chip 5 can be cooled or dissipated, for example, by providing cooling means on the back surface of the flexible substrate 2 or pasting a thermally-conductive flexible material or a heat-dissipative flexible material to the back surface of the flexible substrate 2. In addition to this, cooling or heat dissipation can be further efficiently performed by forming the metal plate on the back surface of the flexible substrate 2.

In a case where the proportion of the area of the metal plate to the area of the flexible substrate 2, there is a decrease in efficiency of radiation of heat generated by the photoirradiation device 1D, as the area of coverage of the flexible substrate 2 with the metal plate is too small. However, this brings an advantage in terms of reduction of the weight of the photoirradiation device 1D. On the other hand, in a case where the proportion of the area of the metal plate to the area of the flexible substrate 2 is large, there is an increase in the weight of the photoirradiation device 1D, as the area of coverage of the flexible substrate 2 with the metal plate is too large. This brings a disadvantage in terms of reduction of the weight. However, this brings about improvement in efficiency of radiation of heat generated by the photoirradiation device 1D.

There is an optimum value of the proportion of the area of coverage of the back surface of the flexible substrate 2 with the metal plate to the area of coverage of the front surface of the flexible substrate 2 with the first, second, and third mounting electrodes 17a, 17b, and 17c. For the purpose of reducing stress applied to the electrical conducting material 16, it is desirable that the proportion be 1. However, for the sake of allowing a certain latitude, the proportion needs only range from 0.5 to 2.

[Modification]

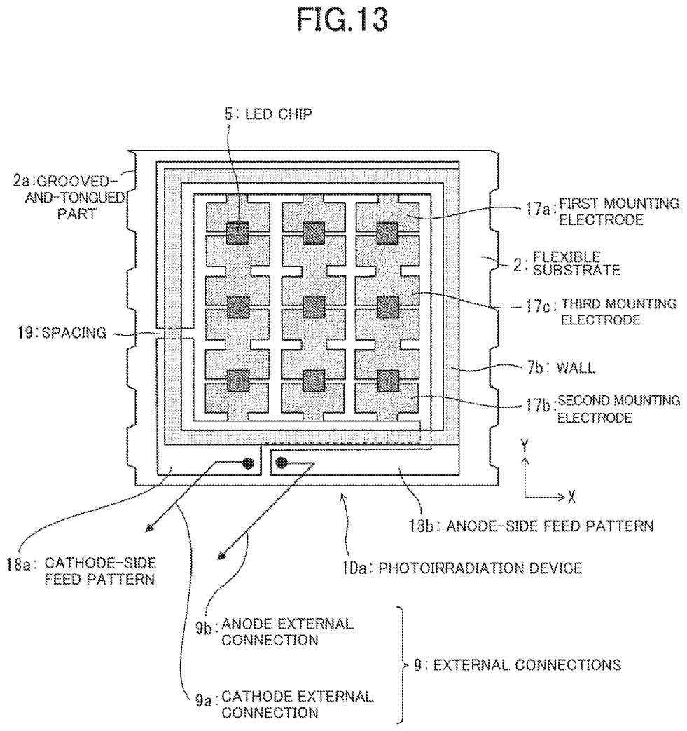

A modification of Embodiment 1 is described with reference to FIG. 13. FIG. 13 is a cross-sectional view showing a configuration of a photoirradiation device 1Da according to the modification.

In the photoirradiation device 1Da, the flexible substrate 2 is formed in a substantially square shape. Further, in the photoirradiation device 1Da, the flexible substrate 2 has grooved-and-tongued parts 2a (joints) formed on two side surfaces of sides thereof extending along the Y direction, respectively. A grooved-and-tongued part(s) 2a may be further formed on one or two side surfaces of a side(s) of the flexible substrate 2 extending along the X direction.

The grooved-and-tongued parts 2a are formed so that tongued portions protruding in the X direction and grooves portions recessed in a direction opposite to the X direction are alternately arranged. Further, the grooved-and-tongued part 2a formed on a first side surface (right in FIG. 13) of the flexible substrate 2 and the grooved-and-tongued part 2a formed on a second side surface (left in FIG. 13) of the flexible substrate 2 have their grooved portions placed on one hand and their tongued portions placed on the other hand in positions corresponding to each other in the X direction. Further, the tongued portions are formed in such shapes and sizes as to be fitted in the grooved portions.

By the flexible substrate 2 thus having the grooved-and-tongued parts 2a, a plurality of the photoirradiation devices 1Da can be joined to each other by the grooved-and-tongued parts 2a. This makes it possible to obtain photoirradiation regions of various sizes and shapes.

Since the photoirradiation device 1Da has its photoirradiation region formed in a defined size at the time of manufacture, the photoirradiation region may be smaller in size than an affected area to be treated and does not conform to the shape of the affected area. In such a case, appropriately joining a plurality of the photoirradiation devices 1Da to each other makes it possible to form a photoirradiation region conforming to the size and shape of an affected area.

As shown in FIG. 13, when the cathode external connection 9a and the anode external connection 9b are on the same side as the mounting electrodes 17b, the cathode-side feed pattern 18a and the anode-side feed pattern 18b are formed along the side surfaces of the flexible substrate 2. It is desirable that the cathode-side feed pattern 18a and the anode-side feed pattern 18b be provided around regions in which the grooved-and-tongued parts 2a are formed.

The cathode-side feed pattern 18a and the anode-side feed pattern 18b are formed by metal. For this reason, when the metal is provided with extension to the regions of formation of the grooved-and-tongued parts 2a, unnecessary protruding objects such as burrs are produced by the metal being also processed when the side surfaces of the flexible substrate 2 are processed to form the grooved-and-tongued parts 2a. Production of such protruding objects constitute an obstacle to joining photoirradiation devices 1Da to each other with the grooved-and-tongued parts 2a. Such inconvenience is caused also in a case where the cathode external connection 9a and the anode external connection 9b are on the same side as the first mounting electrodes 17a.

In the photoirradiation device 1Da, the cathode-side feed pattern 18a and the anode-side feed pattern 18b may be replaced by back-side electrodes 8a and 8b provided on the back side of the flexible substrate 2 as shown in FIGS. 9 and 10. In such a configuration, it is desirable that the first mounting electrodes 17a and the second mounting electrodes 17b be provided around the regions of formation of the grooved-and-tongued parts 2a. This is for the same reason as the reason why the cathode-side feed pattern 18a and the anode-side feed pattern 18b are provided around the regions of formation of the grooved-and-tongued parts 2a.

Further, the present modification provides the grooved-and-tongued parts 2a in order to join a plurality of the photoirradiation devices 1Da to each other. However, a junction structure other than the grooved-and-tongued parts 2a may be used to join a plurality of the photoirradiation devices 1Da to each other. For example, the flexible substrate 2 may have a plurality of hooks provided at intervals on one side surfaces of the two sides thereof extending along the Y direction and have a plurality of holes, provided on the other side surface of the sides, in which the hooks can be engaged.

### CONCLUSION

According to Aspect 1 of the present invention, there is provided a phototherapeutic device including a flexible substrate 2, a plurality of light-emitting elements (LED chips 5) arranged in a matrix over the flexible substrate 2, walls 7, 7a, or 7b having flexibility and surrounding the plurality of light-emitting elements, and a protective resin 10, formed within the walls 7, 7a, or 7b so as to cover the plurality of light-emitting elements, that has translucency to transmit emitted light emitted by the plurality of light-emitting elements and has flexibility. The emitted light is radiated from a region surrounded by the walls 7, 7a, or 7b.

The foregoing configuration makes it possible to, by the protective resin 10 absorbing heat generated by the emitted light from the plurality of light-emitting elements, reduce the heat of light with which an affected area is irradiated. Accordingly, the photoirradiation device does not need to include a complex cooling structure and does not impair the flexibility of the flexible substrate 2. This makes it possible to uniformly irradiate an uneven affected area with light in conformance with the affected area.

According to Aspect 2 of the present invention, there may be provided the phototherapeutic device according to Aspect 1, wherein an average distance between the light-emitting elements adjacent to each other in a first direction falls within a range of 0.5 to 4 times an average distance between the light-emitting element closest to the walls 7, 7a, or 7b in the first direction and the walls 7, 7a, or 7b, and an average distance between the light-emitting elements adjacent to each other in a second direction orthogonal to the first direction falls within a range of 0.5 to 4 times an average distance between the light-emitting element closest to the walls 7, 7a, or 7b in the second direction and the walls 7, 7a, or 7b.

The foregoing configuration makes it possible to improve the in-plane uniformity of intensity of light.

According to Aspect 3 of the present invention, there may be provided the phototherapeutic device according to Aspect 1 or 2, wherein the walls 7, 7a, or 7b have light reflectivity to reflect the emitted light.

The foregoing configuration makes it possible to cause the emitted light from the light-emitting elements to be reflected by the walls 7, 7a, or 7b and extract light through the protective resin 10. Further, the foregoing configuration makes it possible to substantially prevent a region other than the region surrounded by the walls 7, 7a, or 7b from being irradiated with light.

According to Aspect 4 of the present invention, there may be provided the phototherapeutic device according to any one of Aspects 1 to 3, further including a reflector 13, formed over the flexible substrate 2, that reflects light.

The foregoing configuration allows light radiated from the phototherapeutic device to, even if reflected by an affected area to return to the phototherapeutic device, to be reflected toward the affected area by being reflected by the reflector 13. This makes it possible to reduce a loss of light to the minimum.

According to Aspect 5 of the present invention, there may be provided the phototherapeutic device according to any one of Aspects 1 to 4, further including a wavelength conversion sheet (phosphor sheet 15), made of resin and provided on the protective resin 10, that contains a wavelength conversion material that converts a wavelength of the emitted light into a different wavelength.

The foregoing configuration makes it possible to irradiate an affected area with light having a wavelength needed for phototherapy.

According to Aspect 6 of the present invention, there may be provided the phototherapeutic device according to Aspect 5, wherein a plurality of the wavelength conversion sheet are provided so as to overlap each other.

The foregoing configuration makes it possible to vary the emission spectrum of the phototherapeutic device. This makes it possible to select and customize an emission spectrum that is most suitable for therapy.

According to Aspect 7 of the present invention, there may be provided the phototherapeutic device according to any one of Aspects 1 to 6, further including feeder wires (a cathode-side feed pattern 18a and an anode-side feed pattern 18b), formed around the plurality of light-emitting elements over the flexible substrate 2, that supply electric power to the plurality of light-emitting elements.

The foregoing configuration makes it unnecessary to include a feeding electrode on a surface of the flexible substrate 2 opposite to the surface over which the light-emitting elements are provided. This brings a cost advantage.

According to Aspect 8 of the present invention, there may be provided the phototherapeutic device according to any one of Aspects 1 to 7, wherein the flexible substrate 2 is formed in a substantially square shape, and the flexible substrate 2 has, on side surfaces extending along at least two sides thereof, joints (grooved-and-tongued parts 2a) for joining a plurality of the flexible substrates 2 to each other.

The foregoing configuration makes it possible to, by joining a plurality of the flexible substrates to each other with the joints, obtain a photoirradiation region conforming to the size and shape of an affected area.

The present invention is not limited to any of the embodiments described above but may be altered in various ways within the scope of the claims, and an embodiment based on a proper combination of technical means disclosed in different embodiments is encompassed in the technical scope of the present invention. Furthermore, a new technical feature can be formed by a combination of technical means respectively disclosed in embodiments.

### REFERENCE SIGNS LIST

1, 1a, 1A to 1D, 1Aa, 1Ab Photoirradiation device (phototherapeutic device)
2 Flexible substrate
2a Grooved-and-tongued part (joint)
5 LED chip (light-emitting element)
7, 7a, 7b Wall
10 Protective resin
13 Reflector
15 Phosphor sheet (wavelength conversion sheet)
16 Electrical conducting material
18a Cathode-side feed pattern (feeder wire)
18b Anode-side feed pattern (feeder wire)

1. A phototherapeutic device comprising:
a flexible substrate;
a plurality of light-emitting elements arranged in a matrix over the flexible substrate;
walls having flexibility and surrounding the plurality of light-emitting elements;
a protective resin, formed within the walls so as to cover the plurality of light-emitting elements, that has translucency to transmit emitted light emitted by the plurality of light-emitting elements and has flexibility; and
feeder wires, formed on the flexible substrate, that are configured to supply electric power to the plurality of light-emitting elements,
wherein the feeder wires are formed in a square shape around a region surrounding the plurality of light-emitting elements, and
the emitted light is radiated from a region surrounded by the walls.

2. The phototherapeutic device according to claim 1, wherein an average distance between those two of the plurality of light-emitting elements which are adjacent to each other in a first direction falls within a range of 0.5 to 4 times an average distance between that one of the plurality of light-emitting elements which is closest to the walls in the first direction and the walls, and
an average distance between those two of the plurality of light-emitting elements which are adjacent to each other in a second direction orthogonal to the first direction falls within a range of 0.5 to 4 times an average distance between that one of the plurality of light-emitting elements which is closest to the walls in the second direction and the walls.

3. The phototherapeutic device according to claim 1, wherein the walls have light reflectivity to reflect the emitted light.

4. The phototherapeutic device according to claim 1, further comprising a reflector, formed over the flexible substrate, that is configured to reflect the emitted light.

5. The phototherapeutic device according to claim 1, further comprising a wavelength conversion sheet, made of resin and provided on the protective resin, that contains a wavelength conversion material that converts a wavelength of the emitted light into a different wavelength.

6. The phototherapeutic device according to claim 5, wherein a plurality of the wavelength conversion sheet are provided so as to overlap each other.

7. (canceled)

8. The phototherapeutic device according to claim 1, wherein the flexible substrate is formed in a substantially square shape, and
the flexible substrate has, on side surfaces extending along at least two sides thereof, junctions for joining another plurality of flexible substrates to each other.

9. A phototherapeutic device comprising:
a flexible substrate;
a plurality of light-emitting elements arranged in a matrix over the flexible substrate;
walls having flexibility and surrounding the plurality of light-emitting elements;
a protective resin, formed within the walls so as to cover the plurality of light-emitting elements, that has translucency to transmit emitted light emitted by the plurality of light-emitting elements and has flexibility; and
feeder wires that are configured to supply electric power to the plurality of light-emitting elements,
wherein the plurality of light-emitting elements radiate emitted light from a region surrounded by the walls,
the feeder wires are formed on the flexible substrate and include an anode-side feeder wire and a cathode-side feeder wire,
the anode-side feeder wire is formed in an area surrounding three corners of a square region around the plurality of light-emitting elements, and
the cathode-side feeder wire is formed in an area surrounding a remaining one corner of the square region.

10. The phototherapeutic device according to claim 1, wherein a first part of the flexible substrate that adheres to the walls has a surface roughness different from a surface roughness of a second part of the flexible substrate other than the first part.

11. The phototherapeutic device according to claim 1, further comprising, on the protective resin, a resin sheet having translucency and biocompatibility.

12. The phototherapeutic device according to claim 11, wherein the resin sheet has heat insulating properties as well as insulation properties.

13. The phototherapeutic device according to claim 1, further comprising, on the flexible substrate, mounting electrodes on which the plurality of light-emitting elements are mounted,
wherein the walls are formed so that parts of inner edges all around the walls are formed over front surfaces of parts of ones of the mounting electrodes located close to the walls.

14. The phototherapeutic device according to claim 13, wherein the plurality of light-emitting elements are mounted on the mounting electrodes by flip-chip bonding.

15. The phototherapeutic device according to claim 1, wherein the feeder wires include an anode-side feeder wire and a cathode-side feeder wire,
the cathode-side feeder wire has its first end connected to a cathode external connection for connecting to an external power source,
the anode-side feeder wire has its first end connected to an anode external connection for connecting to the external power source, and
the cathode-side feeder wire and the anode-side feeder wire have their second ends facing each other and differing from the cathode external connection and the anode external connection, with a spacing formed between the second ends.

16. The phototherapeutic device according to claim 1, further comprising a metal plate formed on a back surface of the flexible substrate.

17. The phototherapeutic device according to claim 16, further comprising, on the flexible substrate, mounting electrodes on which the plurality of light-emitting elements are mounted,
wherein a proportion of an area of the metal plate to a total of areas of the mounting electrodes ranges from 0.5 to 2.

18. The phototherapeutic device according to claim 17, wherein the proportion is 1.

19. The phototherapeutic device according to claim 1, further comprising a thermally-conductive flexible material pasted to a back surface of the flexible substrate.

20. The phototherapeutic device according to claim 1, further comprising a heat-dissipative flexible material pasted to a back surface of the flexible substrate.

21. The phototherapeutic device according to claim 1, wherein the feeder wires are constituted by a cathode-side feeder wire and an anode-side feeder wire, and
the anode-side feeder wire and the cathode-side feeder wire are separated from each other by a first spacing located on a first side of the square shape and a second spacing located on a second side of the square shape different from the first side.

22. The phototherapeutic device according to claim 21, further comprising an anode external connection and a cathode external connection for connecting to an external power source, the anode external connection and the cathode external connection being connected to an end of the anode-side feeder wire and an end of the cathode-side feeder wire separated from each other by the first spacing, respectively.
